(19) United States
(12) Patent Application Publication
(10) Pub. No.: US 2015/0105801 A1
(43) Pub. Date: Apr. 16, 2015

(54) Foot Cleaning Device

(71) Applicant: Yvonne Antonia Roberts, Bay Minette, AL (US)

(72) Inventor: Yvonne Antonia Roberts, Bay Minette, AL (US)

(21) Appl. No.: 14/054,393

(22) Filed: Oct. 15, 2013

(51) Int. Cl. A61B 17/54; A47K 7/02

(52) U.S. Cl. CPC A61B 17/54; A47K 7/026
USPC 606/131

(57) ABSTRACT

A version of the invention discloses a foot cleaning device. A preferred embodiment of a version of the invention is based on a circular foot cleaning device that uses thin or textured edges to exfoliate and clean the foot and heel. In a preferred embodiment, the device is placed on the foot or heel and rotated. Embodiments of the invention comprise a foot cleaning device that cleans all areas of the foot. Versions of the invention comprise a foot cleaning device for the ball, side, toe area, and heel area of the foot. The invention additionally comprises inserts and attachments. In one embodiment, the inserts and attachments comprise sandstone, pumice stone, or similar materials that allows the user to loosen calluses or dead skin before scraping them off. In another embodiment the inserts and attachments comprise points, spikes, and similar materials that allows the user to loosen calluses or dead skin before scraping them off.

### FIELD OF THE INVENTION

A preferred embodiment of the invention refers to a device for cleaning one's foot or feet.

### BACKGROUND

Exfoliation, the removal of dead skin cells from the skin's surface, is a refreshing step in the skincare process that instantly rejuvenates the skin. While exfoliation is a natural bodily process, dead skin cells often pile up on the skin's surface, creating rough, dull patches. Dermatologists recommend regular exfoliation to keep the skin in optimum condition.

The most common methods of exfoliation are mechanical and chemical exfoliation. Chemical exfoliation utilizes chemicals such as hydroxy acids and enzymes, while manual exfoliation generally uses a tool or exfoliants such as crushed apricot kernels or sugar crystals.

The skin on one's feet is often the driest part of the body, especially for those who wear sandals or flip-flops that expose the feet to air and dust, which can exacerbate dryness. Various aspects of daily life contribute to dry, cracked heels, including cold weather and indoor heating, ill-fitting footwear, improper daily foot care, and standing on your feet for prolonged periods. The heels of the feet are generally more prone to calluses and cracking than other parts of the foot because the feet carry out most bodily movement and the heels receive the brunt of that work. Exfoliation removes the resulting dead skin, calluses, and the smell associated with both, leaving clean and healthy feet.

A number of references disclose a device for cleaning a person's foot, however these disclosures are related to scrubbing and/or filing problem areas from the feet. For example, the product commercially sold as the PedEgg®, USD 596,353, uses a series of sharp blades that grate, rather than scrape, away hard skin. USD 596,353, and other similar devices in the art, use a back-and-forth grating technique that can potentially remove healthy skin from the foot and/or cut the skin, causing discomfort. These unwanted effects are especially likely for those who suffer from peripheral neuropathy, a symptom of which is loss of sensation in the foot. Therefore, a need exists in the art for a foot-cleaning tool based on a 360 degree scraping approach that removes dead skin or calluses on the skin.

### SUMMARY

This Summary is provided to introduce a selection of concepts in a simplified form that are further described below in the Detailed Description. This Summary is not intended to identify key features or essential features of the claimed subject matter nor is this Summary intended to be used to limit the claimed subject matter's scope.

A preferred embodiment of a version of the invention is based on a circular foot-cleaning device that uses various edges to exfoliate and clean the foot and heel. In a preferred embodiment, the device is placed on the foot or heel and rotated. Embodiments of the invention comprise a foot-cleaning device that cleans all areas of the foot. Versions of the invention comprise a foot-cleaning device for the ball, side, toe area, and heel area of the foot. The invention additionally comprises inserts and attachments. In one embodiment, the inserts and attachments comprise sandstone, pumice stone, or similar materials that allows the user to loosen calluses or dead skin before scraping them off. In another embodiment the inserts and attachments comprise points, spikes, and similar materials that allows the user to loosen calluses or dead skin before scraping them off.

### DESCRIPTION OF THE DRAWINGS

These and other features, aspects, and advantages of the present invention will become better understood with regard to the following description, appended claims, and accompanying drawing where:

FIG. 1 depicts a perspective view of an apparatus embodying features of the present foot cleaning device.

FIG. 2 depicts a perspective view of an apparatus embodying features of the present foot cleaning device.

FIG. 3 depicts a perspective view of an apparatus embodying features of the present foot cleaning device.

FIG. 4 depicts a perspective view of an apparatus embodying features of the present foot cleaning device.

FIG. 5 depicts a perspective view of an apparatus embodying features of the present foot cleaning device.

FIG. 6 depicts an expanded view of an apparatus embodying features of the present foot cleaning device.

FIG. 7 depicts a perspective view of an apparatus embodying features of the present foot cleaning device.

FIG. 8 depicts a perspective view of an apparatus embodying features of the present foot cleaning device.

FIG. 9 depicts a cross-sectional view of separate embodiments of an apparatus embodying features of the present foot cleaning device.

### DETAILED DESCRIPTION

In the Summary above and in this Detailed Description, and the Claims below, and in the accompanying drawings, reference is made to particular features, including method steps, of the invention. It is to be understood that the disclosure of the invention in this specification includes all possible combinations of such particular features. For example, where a particular feature is disclosed in the context of a particular aspect or embodiment of the invention, or a particular claim, that feature can also be used, to the extent possible, in combination with/or in the context of other particular aspects of the embodiments of the invention, and in the invention generally.

The term “comprises” and grammatical equivalents thereof are used herein to mean that other components, ingredients, steps, etc. are optionally present. For example, an article “comprising” components A, B, and C can contain only components A, B, and C, or can contain not only components A, B, and C, but also one or more other components, or can contain at least one component chosen from A, B, or C.

Where reference is made herein to a method comprising two ore more defined steps, the defined steps can be carried out in any order or simultaneously (except where the context excludes that possibility), and the method can include one or more other steps which are carried out before any of the defined steps, between two of the defined steps, or after all the defined steps (except where the context excludes that possibility).

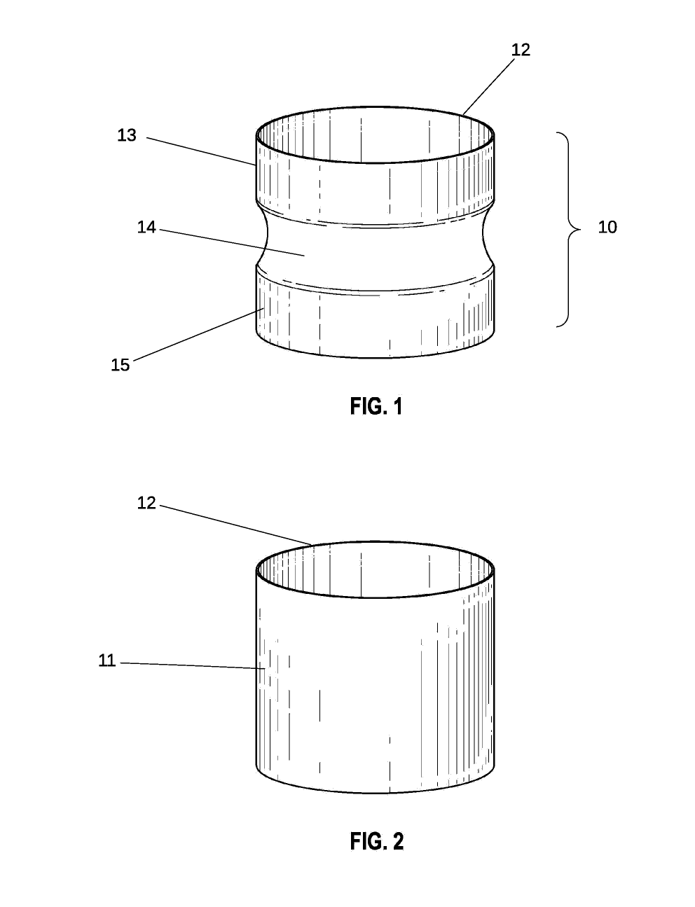

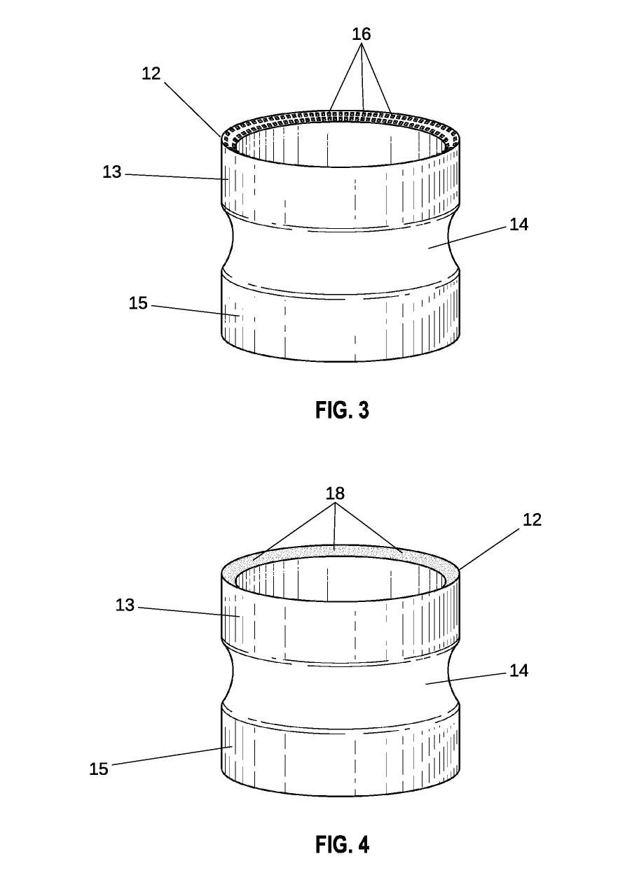

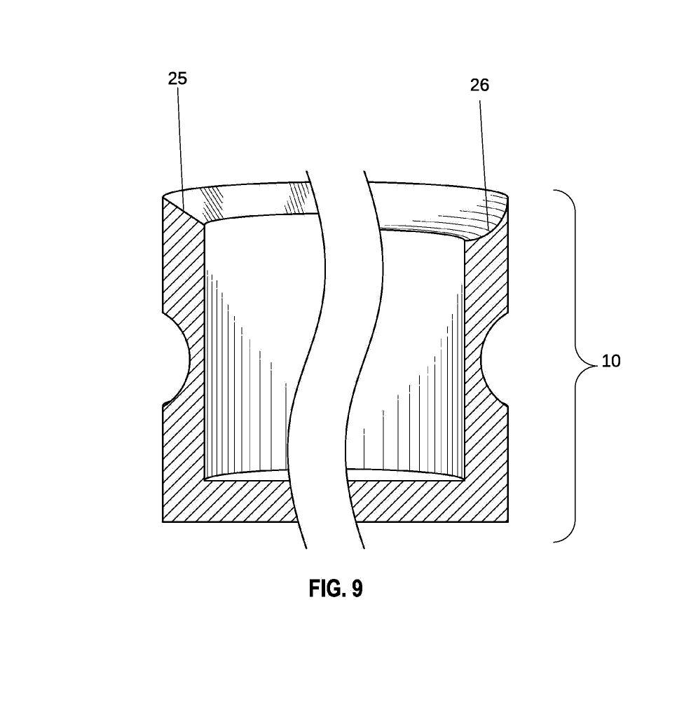

As noted in FIG. 1, a preferred embodiment of a version of the invention is directed to a foot cleaning device 10. As further illustrated in FIG. 2, a preferred embodiment of a version of the invention comprises a tubular body 11 having a foot engagement section 12. In a preferred embodiment, the foot engagement section is a rim, as illustrated in FIG. 2. The rim of the foot engagement section 12 has an edge for removing material from the foot. In one embodiment, as shown in FIG. 2, the rim of the foot engagement section 12 is thin and capable of scraping material from a user's foot. In another embodiment of a version of the invention, the foot engagement section 12 is thicker and slanted, as shown in FIG. 3. As illustrated in FIG. 9, embodiments of a version of the invention may have different slants. As in FIG. 9, one version of the invention provides for a straight slant 25, and another version provides for a curved slant 26.

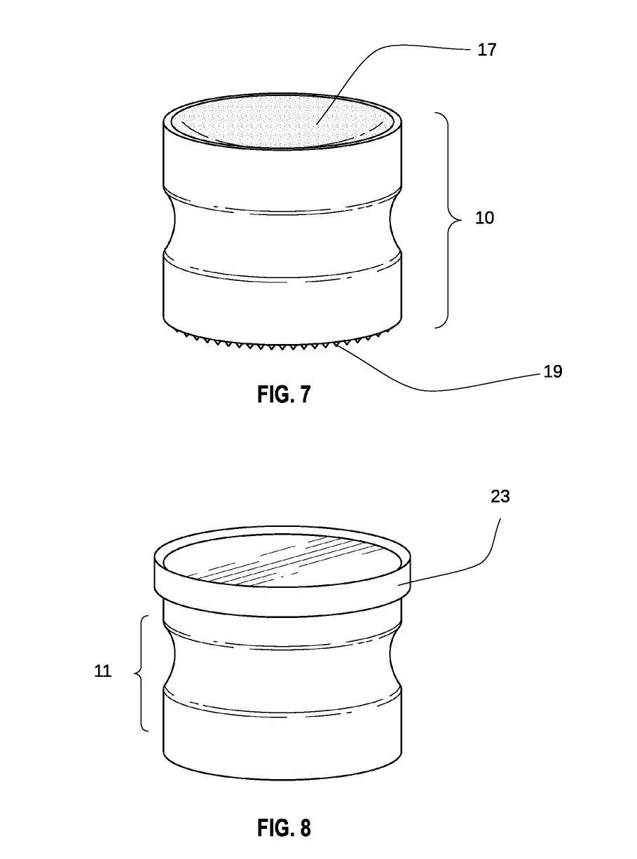

As illustrated in FIG. 1, a foot cleaning device 10 may have an upper 13, middle 14, and lower 15 section. In a preferred embodiment, the upper section 13 makes up at least one foot engagement section 12. In a preferred embodiment, the middle section 14 is indented to form an area for gripping the device 10. In one embodiment of a version of the invention, the lower section 15 is a base. As illustrated in FIG. 6, the base 15 may comprise a separate foot engagement section 12. In a preferred embodiment the base 15 is an insert 17 located on the interior of the tubular body 11. In one embodiment, the insert 17 is a rough surface for creating friction when a user rubs the insert on her/his foot. In yet another embodiment, the insert 17 is an exfoliating material. As illustrated in FIG. 7 an embodiment of a version of the invention comprises a foot cleaning device 10 having an insert 17 and an attachment 19 that allows the user to loosen calluses or dead skin before scraping them off.

As illustrated in FIG. 8, another embodiment of a version of the invention comprises a foot cleaning device having an attached foot engagement section 23. In one embodiment the attached foot engagement section 23 is wider than the tubular body 11.

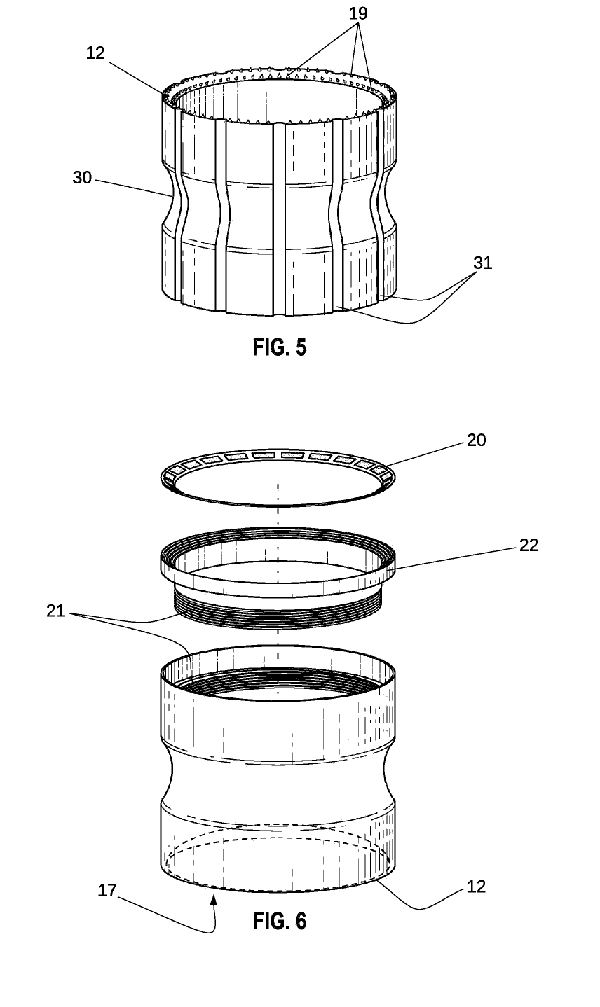

As illustrated in FIG. 1, a preferred embodiment of the invention comprises an indentation in the middle section 14 for gripping the device 10. It is understood that the invention covers indentations or gripping elements placed on any part of the device 10. The gripping elements may be raised elements or indented elements. As further illustrated in FIG. 5, an embodiment of a version of the invention comprises both lateral 30 and horizontal 31 indentations. In a preferred embodiment, the tubular body 11 is shaped to fit a user's heel.

As illustrated in FIG. 3, one embodiment of a version of the invention comprises a tubular body having an upper 13, middle 14, and lower 15 section. The upper section of the tubular body having a foot engagement section 12. As illustrated in FIG. 3, in a preferred embodiment the foot engagement section 12 has attachments 16 for cleaning areas of the foot. As further illustrated in FIG. 3, in one embodiment, the attachments 16 on the foot engagement section are raised surfaces causing friction when the user rubs the attachments on her/his foot.

In yet another embodiment of a version of the invention, as illustrated in FIG. 4, the foot engagement section 12 has an exfoliation attachment 18. In versions of the invention, the exfoliation attachment 18 comprises sandstone, pumice stone, or similar materials that allows the user to loosen calluses or dead skin before scraping them off.

In yet another embodiment of a version of the invention, as illustrated in FIG. 5, the foot engagement section 12 has a pointed attachment 19 comprising points, spikes, and similar materials that allows the user to loosen calluses or dead skin before scraping them off.

As illustrated in FIG. 6, an embodiment of a version of the invention comprises a foot cleaning device having interchangeable attachments 20. In one embodiment, an interchangeable attachment 20 is an exfoliating material. In yet another embodiment, the interchangeable attachment 20 is a thin edge for scraping a user's foot. In yet another embodiment, the interchangeable attachment 20 has raised surfaces for causing friction when rotated on a user's foot.

As further illustrated in FIG. 6, a preferred embodiment of a version of the invention comprises the foot engagement section being threaded 21. The threaded foot engagement section 21 receiving an attachment 22. In one embodiment, the attachment 22 is for cleaning a user's foot. In yet another embodiment, as illustrated in FIG. 6, the attachment 22 secures an interchangeable attachment 20 to the device for cleaning a user's foot. In a preferred embodiment, the foot cleaning device is shaped to fit a user's heel.

A preferred method of cleaning one's heel comprises placing a foot cleaning device 10 on a person's heel such that the foot engagement section 12 is in contact with a person's heel. In a preferred method, the user's foot is wet. In a preferred embodiment, a foot cleaning device 10 is used while bathing or after a shower. Preferably, the foot cleaning device 10 is positioned on a person's heel such that the tubular body 11 of the foot cleaning device 10 is centered on a person's heel. The foot cleaning device 10 is rotated in a circular fashion on the person's heel while applying force. Dead skin and other unwanted materials are removed from a user's heel.

In yet another embodiment, a person cleans her/his heel by using a foot cleaning device 10. In one embodiment, the foot cleaning device 10 has a tubular body 11 having a foot engagement section 12. Preferably, the foot engagement section 12 has a rim. The rim of the foot engagement section 12 has an edge for removing material from the foot. The foot cleaning device 10 is positioned on a person's heel such that the tubular body 11 of the foot cleaning device 10 is centered on a person's heel. The foot cleaning device 10 is rotated in a circular fashion on the person's heel while applying force. In a preferred method, the user's foot is wet. In a preferred embodiment, a foot cleaning device 10 is used while bathing or after a shower. Dead skin and other unwanted materials are removed from a user's heel.

In yet another embodiment, a person cleans her/his heel by using a foot cleaning device 10. In one embodiment, the foot cleaning device 10 has a tubular body 11 having an upper 13, middle 14, and lower 15 portion, The upper portion 13 of the tubular body 11 having a foot engagement section 12. Preferably, the foot engagement section 12 has attachments 16 for cleaning areas of the foot. In other embodiments, the foot engagement section 12 has an exfoliation attachment 18, or an attachment comprising points, spikes, and similar materials 19 that allows the user to loosen calluses or dead skin before scraping them off. In a preferred embodiment, the foot cleaning device 10 is positioned on a person's heel such that the tubular body 11 of the foot cleaning device 10 is centered on a person's heel. The foot cleaning device 10 is rotated in a circular fashion on the person's heel while applying force. Dead skin and other unwanted materials are removed from a user's heel. In a preferred method, the user's foot is wet. In a preferred embodiment, a foot cleaning device 10 is used while bathing or after a shower.

It is understood that versions of the invention may be made from synthetic or natural materials. Versions of the invention may be made of polymers or resins, plastic, rubbers, wood, stone, metal, or other materials.

What is claimed is:

1. A foot cleaning device comprising:
a. A tubular body having a foot engagement section,
b. The foot engagement section having a rim,
c. The rim of the foot engagement section having an edge for removing material from the foot.

2. A foot cleaning device as in claim 1, the device further comprising,
a. The tubular body having an upper, middle, and lower section,
b. The upper section making up the foot engagement section,
c. The middle section being indented to form an area for gripping the device,
d. The lower section being a base.

3. A foot cleaning device as in claim 2, the device further comprising, the base being a second foot engagement section.

4. A foot cleaning device as in claim 3, the device further comprising, the second foot engagement section at the base is an insert located on the interior of the tubular body.

5. A foot cleaning device as in claim 4, the insert being a rough surface for creating friction when a user rubs the insert on her/his foot.

6. A foot cleaning device as in claim 4, the insert being an exfoliating material.

7. A foot cleaning device as in claim 1, the tubular body having indentations for gripping the device.

8. A foot cleaning device as in claim 1, the tubular body shaped to fit a user's heel.

9. A foot cleaning device as in claim 2, the tubular body shaped to fit a user's heel.

10. A foot cleaning device comprising:
a. A tubular body having an upper, middle, and lower portion
b. The upper section of the tubular body having a foot engagement section,
c. The foot engagement section having attachments for cleaning areas of the foot.

11. A foot cleaning device as in claim 10, further comprising, the attachments on the foot engagement section are raised surfaces causing friction when the user rubs the attachments on her/his foot.

12. A foot cleaning device as in claim 10, further comprising, the foot engagement section has a rim having an interchangeable attachment.

13. A foot cleaning device as in claim 12, the interchangeable attachment being an exfoliating material.

14. A foot cleaning device as in claim 12, the interchangeable attachment being a thin edge for scraping a user's foot.

15. A foot cleaning device as in claim 12, the interchangeable attachment having raised surfaces for causing friction when rotated on a user's foot.

16. A foot cleaning device as in claim 10, further comprising,
a. the foot engagement section being threaded.
b. The threaded foot engagement section receiving an attachment,
c. The attachment being for cleaning a user's foot.

17. A foot cleaning device as in claim 10, the tubular body shaped to fit a user's heel.

18. A foot cleaning device as in claim 12, the tubular body shaped to fit a user's heel.

19. The method of cleaning one's heel, the method comprising:
a. Wetting a person's foot,
b. Placing a foot cleaning device on a person's heel such that the foot engagement section is in contact with a person's heel,
c. Positioning the foot cleaning device on a person's heel such that the tubular body of the foot cleaning device is centered on a person's heel,
d. Rotating the foot cleaning device in a circular fashion on the person's heel while applying force,
e. Removing dead skin and other unwanted material from a user's heel.

20. The method of claim 19, further comprising, the foot cleaning device comprises:
a. A tubular body having a foot engagement section,
b. The foot engagement section having a rim,
c. The rim of the foot engagement section having an edge for removing material from the foot.

21. The method of claim 19, further comprising, the foot cleaning device comprises:
a. A tubular body having an upper, middle, and lower portion
b. The upper portion of the tubular body having a foot engagement section,
c. The foot engagement section having attachments for cleaning areas of the foot.
